(19) United States
(12) Patent Application Publication
(10) Pub. No.: US 2007/0218536 A1
(43) Pub. Date: Sep. 20, 2007

(54) Polyvalent Viral Vectors and a System for Production Thereof

(76) Inventors: Guangping Gao, Rosemont, PA (US); James M. Wilson, Gladwyne, PA (US); Xiangyang Zhou, North Wales, PA (US)

Correspondence Address: HOWSON AND HOWSON, SUITE 210, 501 OFFICE CENTER DRIVE, FT WASHINGTON, PA, 19034, US

(21) Appl. No.: 11/587,551

(22) PCT Filed: Apr. 27, 2005
(86) PCT No.: PCT/US05/14485

§ 371 Date: Apr. 17, 2007

Related U.S. Application Data
(60) Provisional application No. 60/565,941, filed on Apr. 28, 2004.

(51) Int. Cl. C12N 15/00; C12N 1/00; C12N 7/01

(52) U.S. Cl. 435/235.1; 435/243; 435/320.1

(57) ABSTRACT

A system for generating viral vectors carrying two distinct expression cassettes is provided. The system utilizes a unique polyvalent transfer vector that permits efficient detection and selection of inserted expression cassettes.

### BACKGROUND OF THE INVENTION

The use of viral vectors to express antigens has been described. Further, the use of fusion peptides as antigen has been described.

Fusion peptide based vectors simplify the dosing regimen and create more opportunities for heterologous boosting. However, the unpredictable nature of fusion peptide processing and epitope presentation, and difficulties in creating and propagating adenoviruses carrying large inserts have become roadblocks to their large-scale applications.

What is needed are predictable methods for generating viral vectors useful for delivery of gene products.

### SUMMARY OF THE INVENTION

A system for generating viral vectors carrying at least two distinct expression cassettes is provided. The system utilizes a unique polyvalent plasmid backbone that permits efficient detection and selection of inserted expression cassettes.

Also provided are methods of generating polyvalent viral particles using the polyvalent backbones of the invention.

These and other embodiments and advantages of the invention are described in more detail below.

### BRIEF DESCRIPTION OF THE DRAWINGS

FIG. 1 illustrates the assembly of a ΔE₁-ΔE₃-adenoviral vectors.

FIG. 2 illustrates the introduction of antigens into the E1 and E3 deletion loci of the adenoviral vectors.

FIG. 3 is a bar chart providing the results of an in vivo comparison of expression from a recombinant chimpanzee C9 adenovirus expressing the same transgene (α1AT) from the E1 locus only (dark bars), or from both the E1 and E3 loci (light bars).

### DETAILED DESCRIPTION OF THE INVENTION

The present invention provides a system for generating a viral vector carrying multiple expression cassettes to a target. The system utilizes a DNA molecule carrying a viral genome containing movable cloning cassettes carrying marker genes. This DNA molecule is used to generate a transfer vector carrying a viral genome that contains multiple heterologous expression cassettes located in different loci within the viral genome. The viral genome carrying the heterologous expression cassettes are rescued from the transfer vector of the invention and packaged in a suitable viral capsid or envelope protein to produce polyvalent viral vectors.

As used herein, the DNA molecule and/or transfer vector can be derived from any genetic element that can carry the viral genome according to the invention and that is capable of transferring the genome into a host cell. Any suitable genetic element (or backbone) can be selected, including, e.g., a plasmid, phage, transposon, cosmid, episome, and the like. In one embodiment, the genetic element is suitable for prokaryotic expression, although other cloning systems can be utilized.

As used herein, the term “different loci” indicates that the heterologous expression cassette for a first selected target product is located in the viral backbone in a site that is non-contiguous with a second heterologous expression cassette, i.e., viral sequences are located between heterologous expression cassettes. These loci may be in different gene regions or in different open reading frames within a single gene region. Alternatively, multiple loci may be within a single open reading frame but non-contiguous with one another, e.g., separated by spacers, native sequences, restriction enzyme sites, or the like.

As used herein an “expression cassette” comprises a nucleic acid sequence encoding a product for delivery to a host cell. The nucleic acid sequence encoding the product is under the control of regulatory control sequences which direct expression of the product in the host. Suitably, the expression cassette is heterologous to the vector sequences flanking the cassette. In one embodiment, the regulatory control elements in each heterologous expression cassette differ from the regulatory control elements in the other heterologous expression cassettes in order to minimize (or eliminate) the risk of homologous recombination during the cloning process and in the viral manipulation process in cells. In one embodiment, each heterologous expression cassette is provided with different promoters and/or enhancers, and/or poly A sequences. However, in other embodiments, one heterologous expression cassette in a polyvalent vector of the invention can have one or more regulatory control element in common with another heterologous expression cassette in the polyvalent vector. In such an embodiment, the regulatory control element is preferably a short sequence, which does not enable recombination.

As described herein, the encoded product may provide a target for the immune system, in order to induce a humoral and/or cellular immune response, may be an adjuvant for another encoded product, may provide an immune modulator effect, and/or may provide a therapeutic effect. Combinations of such products can be delivered in a polyvalent viral vector according to the invention.

The term “functionally deleted” or “functional deletion” means that a sufficient amount of the gene region is removed or otherwise damaged, e.g., by mutation or modification, so that the gene region is no longer capable of producing functional products of gene expression. If desired, the entire gene region may be removed. Other suitable sites for gene disruption or deletion are discussed elsewhere in the application.

I. Polyvalent Viral Construct

A. A DNA Molecule Carrying a Viral Genome and Multiple Reporter Genes

In one aspect, the present invention provides a DNA molecule carrying sequences of a virus which is to be packaged into a polyvalent viral vector. In one embodiment, such a DNA molecule is a plasmid. However, another suitable genetic element, as defined above, may be selected. Introduction of the vector into a cell can be achieved by any means known in the art or as disclosed herein, including transfection.

The viral sequences are selected from the types of virus(es) that are desired for use as a delivery vehicle and that have sufficient space to accommodate multiple expression cassettes. These viral sequences can be readily selected from among viruses having a capsid protein, e.g., adenovirus, or from enveloped viruses, e.g., retroviruses such as feline leukemia virus (FeLV), HTLVI and HTLVII], and lentivirinae [e.g., human immunodeficiency virus (HIV), simian immunodeficiency virus (SIV), feline immunodeficiency virus (FIV), equine infectious anemia virus, and spumavirinal)]), poxviruses (e.g., canarypox), among others. Still other viruses can be readily selected by one of skill in the art.

In one embodiment, the viral sequences are from an adenovirus. Suitably, the polyvalent DNA molecule contains nucleic acid sequences from an adenoviral genome that contains at least the sequences needed to package the viral genome into a capsid. Typically, a polyvalent adenoviral molecule will contain the 5′ adenoviral cis-elements and 3′ adenoviral cis-elements at the extreme 5′ and 3′ termini of the adenoviral genome, respectively. The 5′ end of the adenoviral genome contains the 5′ cis-elements necessary for packaging and replication; i.e., the 5′ inverted terminal repeat (ITR) sequences (which functions as origins of replication) and the 5′ packaging enhancer domains (that contain sequences necessary for packaging linear Ad genomes and enhancer elements for the E1 promoter). The 3′ end of the adenoviral genome includes the 3′ cis-elements (including the ITRs) necessary for packaging and encapsidation.

In addition, the polyvalent DNA molecule may contain additional adenoviral sequences, or may be at least functionally deleted in one or more adenoviral gene regions. In one embodiment, an adenoviral vector used in the invention will contain the E2 region or a functional portion thereof (e.g., the region encoding E2a and/or E2b), and one or more of the late genes, e.g., L1, L2, L3, L4 and L5. In some embodiments, the adenovirus vectors used in the invention may contain all or a portion of the E4 region (e.g., the E4 ORF6).

For example, all or a portion of the adenovirus delayed early gene E3 may be eliminated from the adenovirus sequence which forms a part of the vector. The function of simian E3 is believed to be irrelevant to the function and production of the recombinant virus particle.

For example, an E1-deleted Ad vector can be constructed having a deletion of at least the ORF6 region of the E4 gene, or because of the redundancy in the function of this region, the entire E4 region. Still another vector of this invention contains a deletion in the delayed early gene E2a. Suitably, these vectors retain the late genes (i.e., L1, L2, L3, L4, and L5), and other elements essential for packaging of adenoviral vectors into viral particles. Deletions may also be made in the intermediate genes IX and IVa₂ for some purposes. Other deletions may be made in the other structural or non-structural adenovirus genes. The above discussed deletions may be used individually, i.e., an adenovirus sequence for use in the present invention may contain deletions in only a single region. Alternatively, deletions of entire genes or portions thereof effective to destroy their biological activity may be used in any combination. For example, in one exemplary vector, the adenovirus sequence may have deletions of the E1 genes and the E4 gene, or the E1 genes, with or without deletion of E3, and so on.

In another embodiment, a lentiviral genome is utilized. Typically, a lentiviral vector plasmid contains cis-acting genetic sequences necessary for the vector to infect the target cell and for transfer of the heterologous expression cassettes. Suitably, the original envelop proteins, and gag sequence promoter have been removed.

The viral sequences in the plasmid backbone need not be limited to sequences by the type of capsid or envelope in which they are inserted. Thus, the plasmid backbone may contain viral sequences from one viral source that are encapsidated or packaged into an envelope from another source. For example, a polyvalent HIV vector can be packaged into an FIV envelope; a polyvalent FIV vector can be packaged into an HIV envelope; a polyvalent adenoviral vector can be packaged into a capsid from another serotype. Still other pseudotyped viral vectors will be readily apparent to one of skill in the art.

Once the viral sequences are cloned into a plasmid using techniques known to those of skill in the art, the viral genome is altered to contain a first movable cassette located in a first deleted region of a viral genome and a second movable cassette located in a second deleted region of a viral genome. Optionally, the plasmid can contain multiple movable cassettes, each located in a distinct locus of the viral genome. Each of the movable cassettes is flanked by a unique set of restriction enzyme sites which permits their selective removal from the plasmid and ready insertion of a heterologous expression cassette.

Each the movable cassettes used in the invention contains nucleic acid sequences of a detectable reporter gene operably linked to sequences that will direct expression thereof in a host cell. Suitably, each of the movable cassettes contains a unique reporter gene that is readily distinguishable from reporter genes carried by other movable cassettes carried by the plasmid backbone. In one embodiment, the reporter genes express products which are differentiated from one another by color.

Suitable reporter genes include those encoding products which can be distinguished from other reporter genes carried by the polyvalent plasmid backbone of the invention. For example, fluorescent proteins are distinguishable by color upon excitation with the appropriate wavelength of light, including, e.g., red fluorescent protein, green fluorescent protein, blue fluorescent protein, cyan fluorescent protein, yellow-green fluorescent protein. Suitable fluorescent proteins for use for the selected host cell type are available commercially, e.g., from ClonTech. Still other suitable reporter genes which are distinguishable by color include, e.g., gusA (blue); DsRed (red); luciferase (red); and beta-galactosidase. Alternatively, one of skill in the art can provide another reporter gene that is provided with a tag or label, many of which are known to those of skill in the art.

Suitable reporter genes are selected with a view to the host cell system used for cloning. The host cell itself may be selected from any biological organism, including prokaryotic (e.g., bacterial) cells, and eukaryotic cells, including, insect cells, yeast cells and mammalian cells, as described in more detail below.

In one particularly desirable embodiment, a prokaryotic system is used. Further, the host cell is capable of transfection of DNA and expression of the transfected DNA, and of expressing the selected reporter gene in the manner which is desired, e.g., calorimetrically.

Examples of suitable prokaryotic systems are well known, including bacterial cells. For example, suitable bacterial strains may include, e.g., Escherichia coli C600-F-, e14, mcrA, thr-1 supE44, thi-1, leuB6, lacY1, tonA21, [[lambda]][−] [Huynh, Young, and Davis (1985) DNA Cloning, Vol. 1, 56-110]; DH1-F[−], recA1, endA1, gyrA96, thi-1, hsdR17 (rk[−], mk[+], supE44, relA1, [[lambda]][−] [-Hanahan (1983) J. Mol. Biol. 166, 557-580; XL1Blue-MRF′-D(mcrA)182, D(mcrCB-hsdSMR-mrr)172,endA1, supE44, thi-1, recA, gyrA96, relA1, lac, l-, [F′proAB, lac I[q]ZDM15, Tn10 (tet[r])]; SURE Cells [Stratagene]; e14(mcrA), D(mcrCB-hsdSMR-mrr)171, sbcC, recB, recJ, umuC::Tn5 (kan[r]), uvrC, supE44, lac, gyrA96, relA1, thi-1, end A1[F′proAB, lacI[q]DM15, Tn10(tet[r])]; GM272-F[−], hsdR544 (rk([−], mk[−]), supE44, supF58, lacY1 or [[Delta]]lacIZY6, galK2, galT22, metB1m, trpR55, [[lambda]][−]; HB101-F[−], hsds20 (rb[−], mb[−]), supE44, ara14, galK2, lacY1, proA2, rpsL20 (str[R]), xyl-5, mtl-1, [[lambda]][−], recA13, mcrA(+), mcrB(−) [Raleigh and Wilson (1986) Proc. Natl. Acad. Sci. USA 83, 9070-9074]; JM101-supE, thi, [[Delta]](lac-proAB), [F′, traD36, proAB, lacIqZ[[Delta]]M15], restriction: (rk([+], mk([+]), mcrA+ [Yanisch-Perron et al. (1985) Gene 33, 103-119]; XL-1 blue recA1, endA1, gyrA96, thi, hsdR17 (rk[+], mk[+]), supE44, relA1, [[lambda]][−], lac, [F′, proAB, lacIqZ[[Delta]]M15, Tn10 (tet[R])][-Bullock, et al. (1987) BioTechniques 5, 376-379]; GM2929 [from B. Bachman, Yale E. coli Genetic Stock Center (CSGC#7080)]; M.Marinus strain; sex F[−];(ara-14, leuB6, fhuA13, lacY1, tsx-78, supE44, [glnV44], galK2, galT22, l[−], mcrA, dcm-6, hisG4,[Oc], rfbD1, rpsL136, dam-13::Tn9, xyl-5, mtl-1, recF143, thi-1, mcrB, hsdR2.), MC1000-(araD139, D[ara-leu]7679, gaIU, galK, D[lac]174, rpsL, thi-1); ED8767 (F-,e14-[mcrA],supE44,supF58,hsdS3[rB[−]mB[−]], recA56, galK2, galT22,metB1, lac-3 or lac3Y1. Suitable prokaryotic host cells are available from the American Type Culture Collection, Manassas, Va., US, other public cell depositaries, and a variety of academic and commercial sources. Selection of a suitable cloning system or cell is not a limitation of the present invention.

Each of the movable cassettes used in the construct of the invention is flanked by a unique set of rare restriction enzyme sites. Each set of rare restriction enzyme sites provides a first rare restriction enzyme site at the 5′ end of the movable cassette and a second rare restriction enzyme site the 3′ end of the movable cassette. In one embodiment, the set of rare restriction enzyme sites allows directional cloning of an expression cassette into the locus. However, the invention is not limited to the direction of the insert. In other words, the movable cassette and/or the heterologous expression cassette may be located either 5′ to 3′, or 3′ to 5′ with respect to the orientation of the reading frame of the surrounding viral genome. Further, in certain embodiments, the set of rare restriction enzyme sites may allow non-directional cloning of the expression cassette into the selected locus.

In one example, the rare restriction enzyme I-SceI may be selected for both the 5′ and 3′ rare restriction enzymes sites which compose a single set. This enzyme allows directional cloning, even when flanking both ends of a cassette. In other embodiments, I-SceI may be used in combination with another rare restriction enzyme to form a set of restriction enzyme sites. Suitably, each set of rare restriction enzymes is unique, to allow digestion of only a single locus and ready insertion of a heterologous expression cassette into a selected target site.

In a further embodiment, the rare restriction enzyme is selected so that only the selected location(s) in the viral genome are cleaved, i.e., cleavage is achieved at only the 5′ and 3′ ends of the movable cassette and/or heterologous expression cassette, and neither the genetic element carrying the viral genome or other locations in the viral genome are cut.

In the present application, such a restriction enzyme is termed a rare cutter. Examples of such rare cutters include those having recognitions sites for seven, eight, or more bases, including, e.g., I-Ceu I, PI-Sce I, TevII, BmoI, DmoI, FseI, PacI, PmeI, PsrI, BcgI, BglI, BsabI, BstXI, DrdI, EcoNI, FseI, MaM I, Msl I, Mwo I, Psha I, Sfi I, Swa I, Xcm I, and Xmn I, and the like. Suitable rare cutters may be identified using information readily available to those of skill in the art in the literature and in a variety of on-line databases, e.g., the REBASE™ database. Suitable cutters for the method can be readily determined using a variety of computer programs and/or on-line databases. Suitable restriction enzymes are available from a variety of commercial sources including, e.g., England Biolabs, Obiogene, Lift Technology, Roche, B B Clontech, Stratagene, Amersham Pharmacia, among others.

Thus, the polyvalent plasmid of the invention contains at least two movable cassettes, each of which is flanked by a unique set of rare restriction enzymes that permits selective replacement of the movable cassettes with a heterologous expression cassette. These polyvalent plasmids are transfected into host cells that permit expression of the markers carried by the movable cassettes

B. Polyvalent Transfer Vector Carrying Heterologous Expression Cassettes

Once the appropriate digestion enzyme(s) is selected, conventional digestion and religation techniques are utilized. Typically, the plasmid DNA is mixed with the restriction enzyme(s) and incubated for about 12 to about 48 hours. Following this, a conventional phenol/chloroform extraction step is performed. For example, phenol/chloroform extraction may be utilized, followed by precipitation with ethanol, and dissolving the precipitate (e.g., in TE or another suitable buffer) for use in the remainder of the method steps. See, e.g., Sambrook, Molecular Cloning: A Laboratory Manual, 2^{nd} Ed., 5.28-5.32, Appendix E.3-E.4 (Cold Spring Harbor Press, Cold Spring Harbor, N.Y., 1989). Other suitable methods may be provided by the manufacturer or vendor of the restriction enzyme utilized, or otherwise known to those of skill in the art.

Typically, in order to ensure proper insertion of the heterologous expression cassette, the heterologous expression cassettes is flanked at its 5′ and 3′ end with restriction enzyme restriction sites which are complementary to the set of restriction enzyme sites that flank the movable cassette for the site in which the expression cassettes are inserted.

Thus, typically, a first heterologous expression cassette is cloned into the site of the excised movable cassette. Advantageously, the method of the invention enables the rapid identification of plasmids containing the first heterologous expression cassette. Such plasmids lack expression of the first marker gene, but will be expressing the second marker gene product (as well as any other marker gene products present). In other words, if the first movable cassette expressed green fluorescent protein, the absence of green color following digestion and religation will indicate successful removal of the movable cassette into the site of the first marker gene.

In one embodiment, the digestion and religation steps are repeated sequentially for each of the movable cassettes. In other words, a first digestion step is performed using a first set of restriction enzymes to remove a single movable cassette, in order to ensure that the heterologous expression cassette is inserted in to the desired locus. Thereafter, a second digestion step is performed using a second set of restriction enzyme unique to the set flanking the second movable cassette. A second heterologous expression cassette flanked by restriction enzyme sites corresponding to the set flanking the second movable cassette is ligated into the plasmid backbone and clones are selected which lack expression of the second marker gene. Optionally, one or more further digestion steps are performed in order to remove one or more further movable cassettes.

Thus, the method of the invention permits efficient production of a polyvalent transfer vector useful for production of infectious viral particles.

II. Method of Producing Polyvalent Viral Vector

A polyvalent transfer vector of the invention can be used to generate viral particles, having a capsid or an envelope, using methods known to those of skill in the art. Such techniques include conventional cloning techniques of cDNA such as those described in texts [Sambrook et al., cited above], use of overlapping oligonucleotide sequences of the adenovirus genomes, polymerase chain reaction, and any suitable method which provides the desired nucleotide sequence. Standard transfection and co-transfection techniques are employed, e.g., CaPO₄ precipitation techniques. Other conventional methods employed include homologous recombination of the viral genomes, plaquing of viruses in agar overlay, methods of measuring signal generation, and the like.

Suitable production cell lines are readily selected by one of skill in the art. For example, a suitable host cell can be selected from any biological organism, including prokaryotic (e.g., bacterial) cells, and eukaryotic cells, including, insect cells, yeast cells and mammalian cells. Host cells can be selected from among any mammalian species, including, without limitation, cells such as A549, WEHI, 3T3, 10T1/2, HEK 293 cells or PERC6 (both of which express functional adenoviral E1) [Fallaux, F J et al., (1998), Hum Gene Ther, 9:1909-1917], Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

Generally, when delivering the polyvalent transfer vector comprising the heterologous expression cassettes to a host cell by transfection, the backbone is delivered in an amount from about 5 μg to about 100 μg DNA, or about 10 to about 50 μg DNA to about 1×10⁴ cells to about 1×10¹³ cells, or about 10⁵ cells. However, the relative amounts of plasmid DNA to host cells may be adjusted, talking into consideration such factors as the selected vector, the delivery method and the host cells selected.

Typically, the polyvalent transfer vectors are cultured in the host cells which express the capsid protein and/or envelope protein. In the host cells, the polyvalent viral genomes expressing the heterologous expression cassettes are rescued and packaged into the capsid protein or envelope protein to form an infectious viral particle.

A. Viral Vectors Having Capsid Proteins

In one embodiment, the invention provides a method of packaging a polyvalent viral genome into an infectious viral capsid.

In one embodiment, the viral capsid is derived from an adenovirus. An adenoviral particle or vector of the present invention is composed of an infectious adenovirus protein capsid having packaged therein a polyvalent viral genome containing two or more heterologous expression cassettes, each of these cassettes carrying a product to be expressed in the host. In a further embodiment, these adenoviral vectors are replication-defective, thereby avoiding replication in a host cell.

The selection of the serotype of the adenoviral sequences present in vector is not a limitation of the present invention. A variety of adenovirus strains are available from the American Type Culture Collection, Manassas, Va., or available by request from a variety of commercial and institutional sources. Further, the sequences of many such strains are available from a variety of databases including, e.g., PubMed™ and GenBank™. Homologous adenovirus vectors prepared from other simian or from human adenoviruses are described in the published literature [see, for example, U.S. Pat. No. 5,240,846]. The DNA sequences of a number of adenovirus types are available from GenBank, including type Ad5 [GenBank™ Accession No. M73260]. The adenovirus sequences may be obtained from any known adenovirus serotype, such as serotypes 2, 3, 4, 7, 12 and 40, and further including any of the presently identified human types. Similarly adenoviruses known to infect non-human animals (e.g., simians) may also be employed in the vector constructs of this invention. In one embodiment, at least one of the adenoviruses used in the invention is derived from a non-human primate. Examples of suitable non-human primate sequences including simian adenoviruses, such as, Pan5 (also C5), Pan6 (also C6), Pan7 (also C7), Pan 9 (also C68) and C1. Recombinant adenoviruses have been described for delivery of molecules to host cells. See, U.S. Pat. No. 6,083,716, which provides adenoviral vectors derived from the two chimpanzee adenoviruses, C1 and C68 (also termed Pan 9) and International Patent Publication No. WO 02/33645 [Pan 5, Pan6, Pan7-derived vectors]. However, the invention is not so limited.

A variety of production methods for adenoviral particles is known to those of skill in the art. The selection of appropriate production methods are not a limitation of the present invention. See, e.g., U.S. Pat. No. 6,083,716; International Patent Publication No. WO 02/33645; and U.S. patent application Ser. No. 10/465,302 and its international counterpart, WO 2005/001103.

Briefly, a polyvalent adenoviral transfer vector of the invention that lacks the ability to express a functional version of any essential adenoviral gene product (e.g., E1a, E1b, E2a, E2b, and/or E4 ORF6) can be cultured in the presence of the missing adenoviral gene products which are required for viral infectivity and propagation of an adenoviral particle. These helper functions may be provided by culturing the backbone in the presence of one or more helper constructs (e.g., a plasmid or virus) or a packaging host cell. See, for example, the techniques described for preparation of a “minimal” human adenovirus (Ad) vector in International Patent Publication No. WO96/13597, published May 9, 1996.

1. Helper Viruses

Thus, depending upon the adenovirus gene content of the polyvalent transfer vector employed to carry the expression cassettes, a helper adenovirus or non-replicating virus fragment may be necessary to provide sufficient adenovirus gene sequences necessary to produce an infective recombinant viral particle containing the expression cassette. Useful helper viruses contain selected adenovirus gene sequences not present in the adenovirus vector construct and/or not expressed by the packaging cell line in which the vector is transfected. In one embodiment, the helper virus is replication-defective and contains a variety of adenovirus genes in addition to the sequences described above. Such a helper virus is desirably used in combination with an E1-expressing cell line.

Helper viruses may also be formed into poly-cation conjugates as described in Wu et al., J. Biol. Chem., 264:16985-16987 (1989); K. J. Fisher and J. M. Wilson, Biochem. J, 299:49 (Apr. 1, 1994). Helper virus may optionally contain a second reporter expression cassette. A number of such reporter genes are known to the art. The presence of a reporter gene on the helper virus which is different from the gene product on the adenovirus vector allows both the Ad backbone vector and the helper virus to be independently monitored. This second reporter is used to enable separation between the resulting recombinant virus and the helper virus upon purification.

2. Complementation Cell Lines

To generate recombinant adenoviruses (Ad) deleted in any of the genes described above, the function of the deleted gene region, if essential to the replication and infectivity of the virus, must be supplied to the recombinant virus by a helper virus or cell line, i.e., a complementation or packaging cell line. In many circumstances, a cell line expressing the human E1 can be used to transcomplement the chimp Ad vector. This is particularly advantageous because, due to the diversity between the chimp Ad sequences of the invention and the human AdE1 sequences found in currently available packaging cells, the use of the current human E1-containing cells prevents the generation of replication-competent adenoviruses during the replication and production process. However, in certain circumstances, it will be desirable to utilize a cell line that expresses the E1 gene products for production of an E1-deleted simian adenovirus. Such cell lines have been described. See, e.g., U.S. Pat. No. 6,083,716.

If desired, one may utilize the sequences provided herein to generate a packaging cell or cell line that expresses, at a minimum, the adenovirus E1 gene under the transcriptional control of a promoter for expression in a selected parent cell line. Inducible or constitutive promoters may be employed for this purpose. Examples of such promoters are described in detail elsewhere in this specification. A parent cell is selected for the generation of a novel cell line expressing any desired Ad gene. Without limitation, such a parent cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75], among others. These cell lines are all available from the American Type Culture Collection, 10801 University Boulevard, Manassas, Va. 20110-2209. Other suitable parent cell lines may be obtained from other sources.

Such E1-expressing cell lines are useful in the generation of recombinant adenovirus E1 deleted vectors. Additionally, or alternatively, the invention provides cell lines that express one or more simian adenoviral gene products, e.g., E1a, E1b, E2a, and/or E4 ORF6, can be constructed using essentially the same procedures for use in the generation of recombinant simian viral vectors. Such cell lines can be utilized to transcomplement adenovirus vectors deleted in the essential genes that encode those products. The preparation of a host cell according to this invention involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus genomes, combined with polymerase chain reaction, synthetic methods, and any other suitable methods which provide the desired nucleotide sequence.

In still another alternative, the essential adenoviral gene products are provided in trans by a vector and/or helper virus. In such an instance, a suitable host cell can be selected from any biological organism, including prokaryotic (e.g., bacterial) cells, and eukaryotic cells, including, insect cells, yeast cells and mammalian cells. Suitable host cells include those known in the art, as well as those identified herein.

3. Assembly of Viral Particle and Transfection of a Cell Line

One or more of the missing adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently.

Furthermore, the promoters for each of the adenoviral genes may be selected independently from a constitutive promoter, an inducible promoter or a native adenoviral promoter. The promoters may be regulated, for example, by a specific physiological state of the organism or cell (i.e., by the differentiation state or in replicating or quiescent cells) or by exogenously-added factors. Introduction of the molecules (as plasmids or viruses) into the host cell may also be accomplished using techniques known to the skilled artisan and as discussed throughout the specification. In one embodiment, direct cloning techniques are utilized. Such techniques have been described [G. Gao et al., Gene Ther. 2003 October; 10(22):1926-1930; US Patent Publication No. 2003-0092161-A, May 15, 2003; International Patent Application No. PCT/US03/12405]. In another embodiment, standard transfection techniques are used, e.g., CaPO₄ transfection or electroporation. Assembly of the selected DNA sequences of the adenovirus (as well as the sequences encoding the product and other vector elements) into various intermediate plasmids, and the use of the plasmids and vectors to produce a recombinant viral particle are all achieved using conventional techniques. For example, following the construction and assembly of the desired polyvalent viral vector, the vector is transfected in vitro in the presence of a helper virus into the packaging cell line. Homologous recombination occurs between the helper and the vector sequences, which permits the adenovirus-gene sequences in the vector to be replicated and packaged into virion capsids, resulting in the recombinant viral vector particles. The current method for producing such virus particles is transfection-based. However, the invention is not limited to such methods.

The resulting recombinant adenoviruses are useful in transferring two or more selected heterologous expression cassettes to a selected cell.

B. Viral Vectors Having Envelope Proteins

In another embodiment, the transfer vectors of the invention are used to package a viral vector into an infectious particle of a virus having an envelope protein, e.g, a lentivirus or a poxvirus. Examples of suitable lentiviruses include, e.g., human immunodeficiency virus (HIV), simian immunodeficiency virus (SIV), caprine arthritis and encephalitis virus, equine infectious anemia virus, bovine immunodeficiency virus, visna virus, and feline immunodeficiency virus (FIV). The examples provided herein illustrate the use of minigenes derived from HIV and FIV. However, other lentiviruses of human or non-human origin may also be used. The sequences used in the constructs of the invention may be derived from academic, non-profit (e.g., the American Type Culture Collection, Manassas, Va.) or commercial sources of lentiviruses.

Methods of generating such viral vectors are known to those of skill in the art.

Methods of producing lentiviral vectors have been described. See, e.g., J E Coleman, et al., Physiol Genomics. 2003 Feb. 6; 12(3):221-8., which described production of an HIV-1 based lentiviral system using self-inactivating lentiviral vectors. In one example, lentiviral vectors are created in a transient transfection system in which a cell line is transfected with three separate plasmid expression systems. These include the transfer vector plasmid, which is produced according to the present invention and contains portions of the selected lentiviral provirus and the heterologous expression cassettes, the packaging plasmid or construct, and a plasmid with a lentiviral envelope gene (ENV) which may be an envelope protein of the same lentivirus or a different virus [Amado and Chen, Lentiviral Vectors—the Promise of Gene Therapy Within Reach? Science. 285 (5428): 674-76 (1999)]. The three plasmid components of the vector are put into a packaging cell which is then inserted into a lentiviral shell.

In one embodiment, the transfer vector plasmid contains cis-acting genetic sequences necessary for the vector to infect the target cell and for transfer of the therapeutic (or reporter) gene and contains restriction sites for insertion of desired genes. The 3′ and 5′ LTRs, the original envelope proteins, and gag sequence promoter have been removed. The packaging plasmid contains the elements required for vector packaging such as structural proteins, HIV genes (except the gene env which codes for infection of T cells, or the vector would only be able to infect these cells), and the enzymes that generate vector particles. Typically, the packaging signals and their adjacent signals are removed so the parts responsible for packaging the viral DNA have been separated from the parts that activate them. Thus, the packaging sequences will not be incorporated into the viral genome and the virus will not reproduce after it has infected the host cell.

The third plasmid's envelope gene of a different virus specifies what type of cell to target and infect instead of the T cells, e.g., the glycoprotein of vesicular stomatitis virus, known as VSV, MLV, among others. Normally HIV can infect only helper T-cells because they use their gp120 protein to bind to the CD4 receptor. However, it is possible to genetically exchange the CD4 receptor-binding protein for another protein that codes for the different cell type on which gene therapy will be performed. This gives the lentiviral vector a broad range of possible target cells. Other lentiviral production methods and vector elements are described in International Patent Publication No. WO 03/092582, which is incorporated by reference.

Still other enveloped viral vectors can be produced using the polyvalent viral backbones methods of the invention. See, e.g. “The Uses of Poxviruses as Vectors”, Current Gene Therapy, vol. 3, no. 6, pp. 583-595 (December 2003); M. E. Perkus, et al., “Poxvirus-based vaccine candidates for cancer, AIDS, and other infectious diseases”, Journal of Leukocyte Biology, Vol 58, Issue 1 1-13, (1995).

III. Uses for Polyvalent Viral Vector

The polyvalent viral vectors of the invention are formulated in a composition containing a physiologically compatible carrier. These compositions are useful in a variety of therapeutic and immunization regimens. Advantageously, the expression of multiple gene products from the polyvalent viral vectors may reduce the amount of vector, or other drug, necessary to deliver to the subject to achieve the desired biological effect.

In one embodiment, the polyvalent viral vector of the invention contains a heterologous expression cassette carrying a therapeutic product. Such a polyvalent viral vector can further carry one or more additional therapeutic products. An additional therapeutic product can be directed to treatment the same conditions or symptoms related to the first therapeutic product, or to a different indication. Where desired, a selected therapeutic gene product can be delivered to modulate any reaction to the polyvalent viral vector.

In another embodiment, the polyvalent viral vector of the invention contains a heterologous expression cassette carrying a product which induces a humoral and/or cytotoxic immune response to a target. Such a polyvalent viral vector can further carry one or more additional such immunogenic products. This second product can be directed to inducing an immune response to the target, or a cross-reactive target. In another embodiment, the second product can be a therapeutic product designed to treat symptoms associated with the underlying condition. In still another embodiment, the second product can be an adjuvant for another gene product delivered by the polyvalent viral vector. In yet another embodiment, the second product is a therapeutic product delivered to modulate any reaction to the polyvalent viral vector.

As used herein, immune modulators include products that modify the reaction of the immune system, e.g., to a viral vector. Examples of immune modulators include, e.g., cytokines and interleukins.

As used herein, suitable immunomodulatory compounds can include, e.g., CTLA4 immunoglobulin; anti-CD4 antibodies; FK506; and interleukins (IL), including any of IL1-21, e.g., IL-2, IL-3, IL-4, IL-10, IL-12, and IL-18. For example, IL-10 may be useful in down-modulating a local anti-inflammatory response; Fas ligand may be useful in down-regulating adenovirus-mediated T cell responses.

Still other suitable combinations of products to be delivered in a polyvalent viral vector of the invention, in a cocktail containing one or more polyvalent viral vectors of the invention, or in a regimen involving delivery of one or more polyvalent viral vectors of the invention, will be apparent to those of skill in the art from the present specification.

A. Polyvalent Viral Vector-Mediated Delivery of Therapeutic Molecules

In one embodiment, the polyvalent vectors are administered to humans according to published methods for gene therapy. A viral polyvalent vector carrying multiple heterologous expression control cassettes may be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The polyvalent vectors are administered in sufficient amounts to transduce the target cells and to provide sufficient levels of gene transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the retina and other intraocular delivery methods, direct delivery to the liver, inhalation, intranasal, intravenous, intramuscular, intratracheal, subcutaneous, intradermal, rectal, oral and other parenteral routes of administration. Routes of administration may be combined, if desired, or adjusted depending upon the gene product or the condition. The route of administration primarily will depend on the nature of the condition being treated.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the therapeutic product, or for an immunogen, the level of circulating antibody, can be monitored to determine the frequency of dosage administration. Yet other methods for determining the timing of frequency of administration will be readily apparent to one of skill in the art.

In one embodiment, a polyvalent viral vector contains a heterologous expression cassette encoding a therapeutic product and a heterologous expression cassette encoding an immune modulator. The selected immune modulator is defined herein as an agent capable of inhibiting the formation of neutralizing antibodies directed against the recombinant vector of this invention or capable of inhibiting cytolytic T lymphocyte (CTL) elimination of the vector. The immune modulator may interfere with the interactions between the T helper subsets (T_{H1} or T_{H2}) and B cells to inhibit neutralizing antibody formation. Alternatively, the immune modulator may inhibit the interaction between T_{H1} cells and CTLs to reduce the occurrence of CTL elimination of the vector. A variety of useful immune modulators and dosages for use of same are described, for example, in Yang et al., J. Virol., 70(9) (September, 1996); International Patent Publication No. WO 96/12406, published May 2, 1996; and International Patent Publication No. WO 96/26285.

1. Therapeutic Product

Useful therapeutic products encoded by the heterologous expression cassette include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGF α), platelet-derived growth factor (PDGF), insulin growth factors I and II (IGF-I and IGF-II), any one of the transforming growth factor superfamily, including TGF, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful gene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors and, interferons, and, stem cell factor, flk-2/flt3 ligand. Gene products produced by the immune system are also useful in the invention. These include, without limitation, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target. Reduction and/or modulation of expression of a gene are particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

The polyvalent viral vectors of the invention are particularly well suited for therapeutic regimens in which multiple viral-mediated deliveries of gene products is desired, e.g., in regimens involving redelivery of the same product or in combination regimens involving delivery of other genes products.

B. Polyvalent Viral Mediated Delivery of Immunogenic Gene Products

The polyvalent viral vectors of the invention may also be employed as immunogenic compositions. As used herein, an immunogenic composition is a composition to which a humoral (e.g., antibody) or cellular (e.g., a cytotoxic T cell) response is mounted to a product delivered by the immunogenic composition following delivery to a mammal, and preferably a primate. The present invention provides a polyvalent viral vector that can contain in any of its adenovirus sequence deletions a first heterologous expression cassette encoding a desired immunogen. The polyvalent viral vector can further contain a heterologous expression cassette encoding an adjuvant for the immunogen, additional immunogenic products, a therapeutic product, or a product that down-regulates an immune response to the polyvalent viral vector.

When the polyvalent viral vector is a polyvalent adenoviral vector, suited for use as a live recombinant virus vaccine in different animal species compared to an adenovirus of human origin, but is not limited to such a use. The recombinant polyvalent viruses can be used as prophylactic or therapeutic vaccines against any pathogen for which the antigen(s) crucial for induction of an immune response and able to limit the spread of the pathogen has been identified and for which the cDNA is available.

Such vaccinal (or other immunogenic) compositions are formulated in a suitable delivery vehicle, as described above. Generally, doses for the immunogenic compositions are in the range defined above for therapeutic compositions. The levels of immunity of the selected gene can be monitored to determine the need, if any, for boosters. Following an assessment of antibody titers in the serum, optional booster immunizations may be desired.

Optionally, a vaccinal composition of the invention may be formulated to contain other components, including, e.g. adjuvants, stabilizers, pH adjusters, preservatives and the like. Such components are well known to those of skill in the vaccine art. Examples of suitable adjuvants include, without limitation, liposomes, alum, monophosphoryl lipid A, and any biologically active factor, such as cytokine, an interleukin, a chemokine, a ligands, and optimally combinations thereof. Certain of these biologically active factors can be expressed in vivo, e.g., via a plasmid or viral vector. For example, such an adjuvant can be administered with a priming DNA vaccine encoding an antigen to enhance the antigen-specific immune response compared with the immune response generated upon priming with a DNA vaccine encoding the antigen only.

The polyvalent viral vectors are administered in a “an immunogenic amount”, that is, an amount of polyvalent viral vector is effective in a route of administration to transfect the desired cells and provide sufficient levels of expression of the selected gene to induce an immune response. Where protective immunity is provided, the polyvalent viral vectors are considered to be vaccine compositions useful in preventing infection and/or recurrent disease.

Alternatively, or in addition, the vectors of the invention may contain a gene encoding a peptide, polypeptide or protein which induces an immune response to a selected immunogen. The polyvalent adenoviral vectors of this invention are expected to be highly efficacious at inducing cytolytic T cells and antibodies to the inserted heterologous antigenic protein expressed by the vector.

For example, immunogens may be selected from a variety of viral families. Examples of viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C [see, e.g., US Published Patent Application No. US 2003/190606 (Oct. 9, 2003); US 2002/081568 (Jun. 27, 2002)] or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis, and the putative causative agent of sudden acute respiratory syndrome (SARS). Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. [See, U.S. Pat. No. 5,891,994 and U.S. Pat. No. 6,193,981.] See, also, the HIV and SIV proteins described in D. H. Barouch et al., J. Virol., 75(5):2462-2467 (March 2001), and R. R. Amara, et al., Science, 292:69-74 (6 Apr. 2001). In another example, the HIV and/or SIV immunogenic proteins or peptides may be used to form fusion proteins or other immunogenic molecules. See, e.g., the HIV-1 Tat and/or Nef fusion proteins and immunization regimens described in International Patent Publication No. WO 01/54719, published Aug. 2, 2001, and International Patent Publication No WO 99/16884, published Apr. 8, 1999. The invention is not limited to the HIV and/or SIV immunogenic proteins or peptides described herein. In addition, a variety of modifications to these proteins has been described or could readily be made by one of skill in the art. See, e.g., the modified gag protein that is described in U.S. Pat. No. 5,972,596. Further, any desired HIV and/or SIV immunogens may be delivered alone or in combination. Such combinations may include expression from a single vector or from multiple vectors.

Optionally, another combination may involve delivery of one or more expressed immunogens with delivery of one or more of the immunogens in protein form. Such combinations are discussed in more detail below. The papovavirus family includes the sub-family polyomaviruses (BKU and JCU viruses) and the sub-family papillomavirus (associated with cancers or malignant progression of papilloma). For example, papillomavirus antigens and combinations thereof have been described. See, e.g., US Published Application No. 2003/129199 (Jul. 10, 2003); US Published Application No. 2002/18221 (Dec. 15, 2002); U.S. Pat. No. 6,342,224.

The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxyirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxyirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Heath and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including cytotoxic T-lymphocytes (CTLs) to eliminate those T cells. In rheumatoid arthritis (RA), several specific variable regions of T-cell receptor (TCRs) which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a recombinant simian adenovirus that encodes at least one of these polypeptides will elicit an immune response that will target T cells involved in scleroderma.

The following examples illustrate the cloning of the polyvalent adenoviruses and the construction of exemplary polyvalent adenovirus vectors of the present invention. These examples are illustrative only, and do not limit the scope of the present invention.

### EXAMPLES

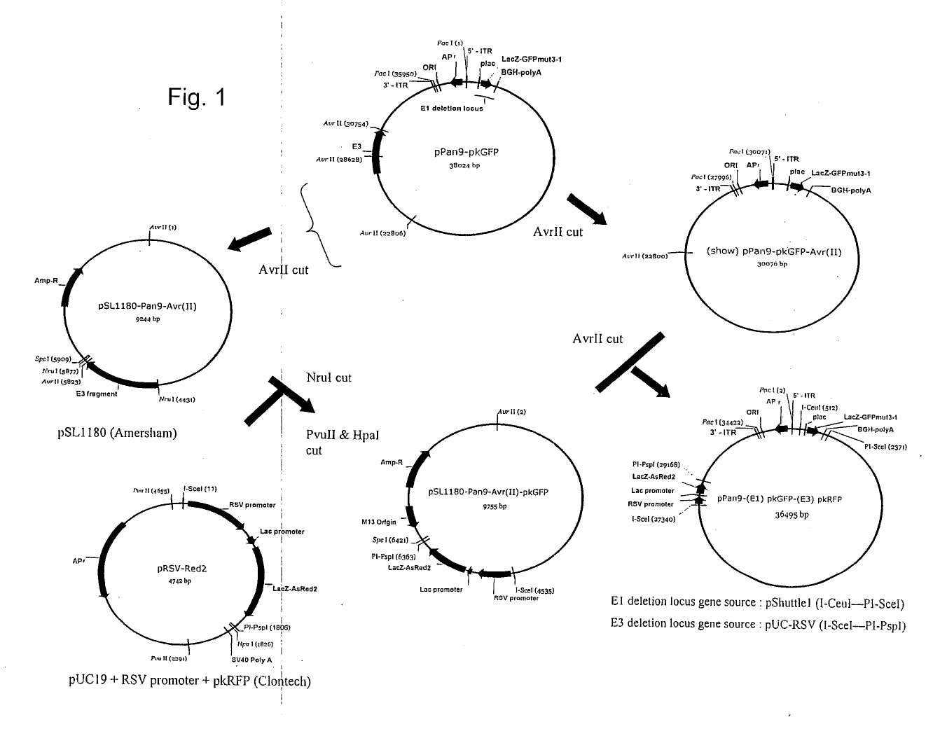

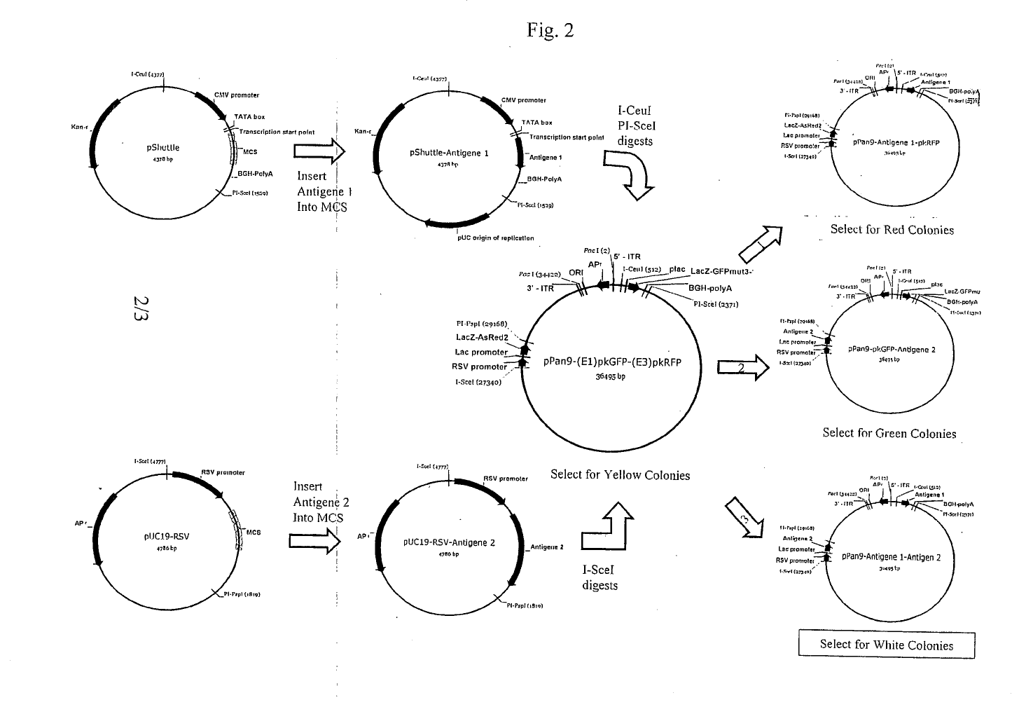

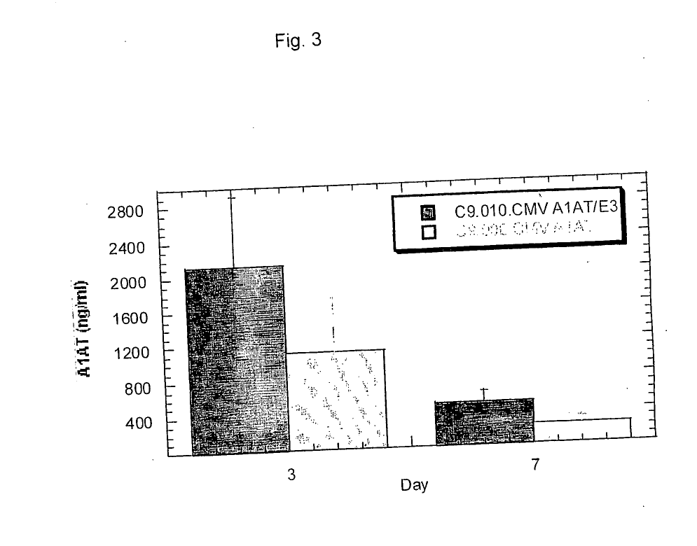

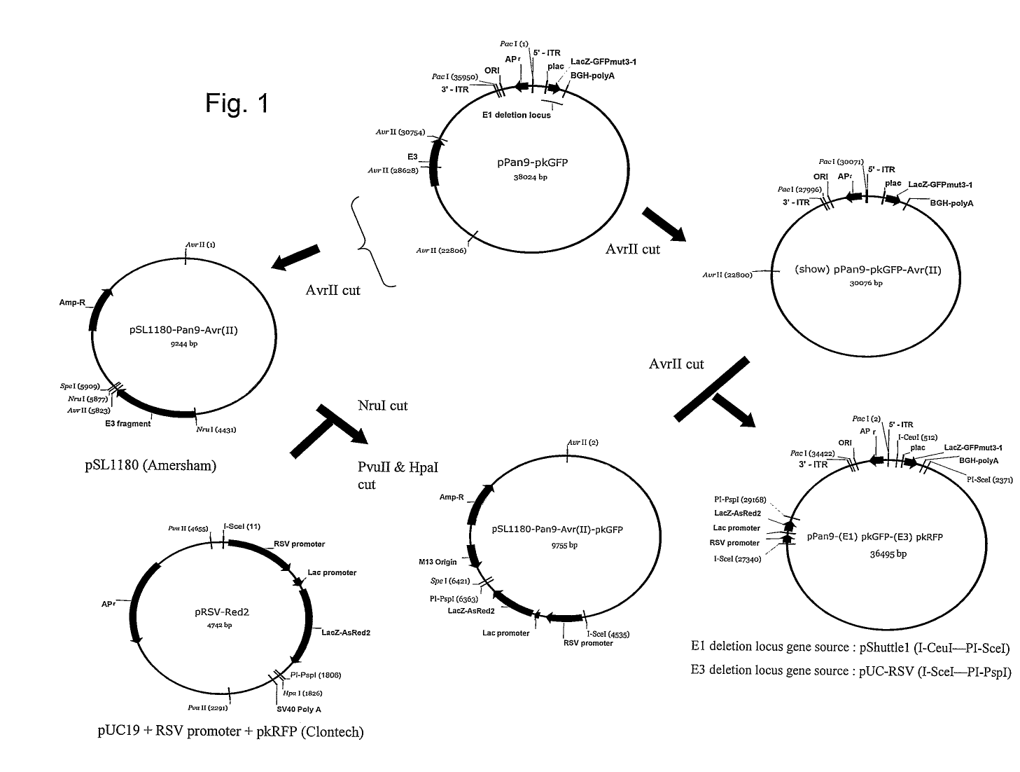

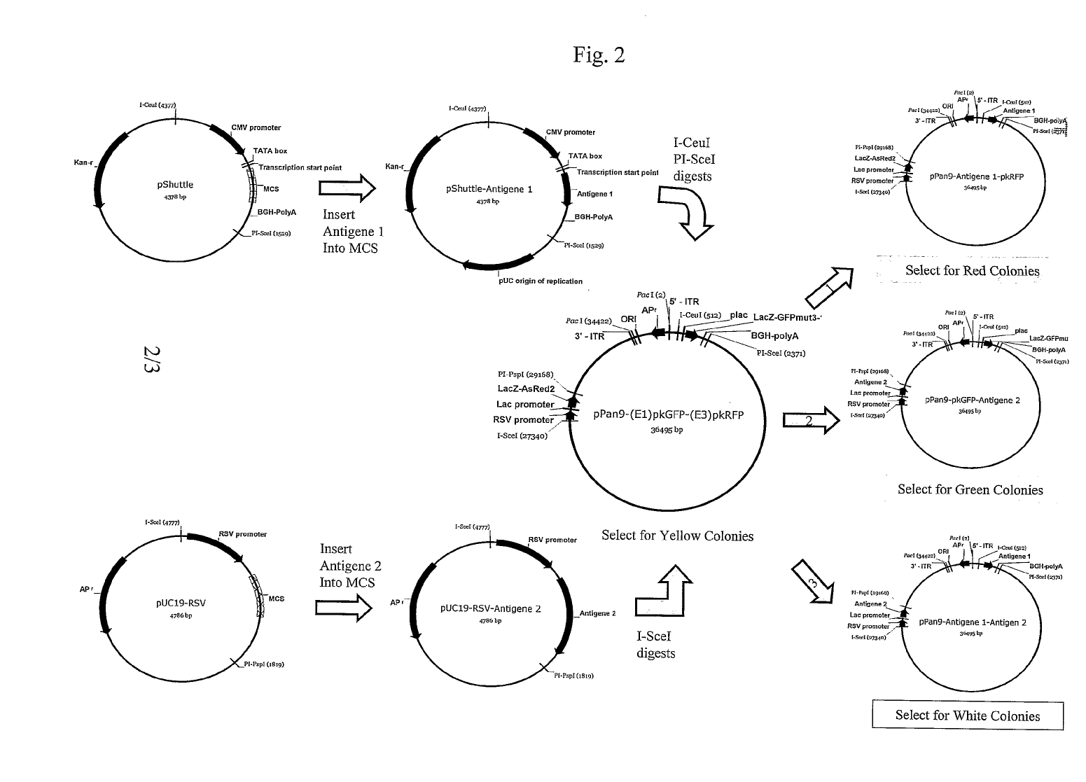

As illustrated if FIGS. 1 and 2, chimpanzee adenovirus Pan9 based vector is constructed as a polyvalent transfer vector according to the invention.

A. Construction of DNA Molecule with Movable Cassettes

With reference to FIG. 1, a plasmid backbone was selected which carries the chimpanzee adenovirus Pan9 viral genome, having a mutant green fluorescent protein marker (GFP) cassette in the site of the Pan9 E1 gene region, the bovine growth hormone polyA, and a partial E3-deletion. This plasmid is described in more detail in International Patent Publication No. WO 2003/046124.

pPan9-pkGFP is digested with AvrII (FIG. 1A) to remove the E3 region of Pan 9 which is cloned into pSL1180 [obtained commercially from Pharmacia], to form pSL1180-Pan9-Avr(II), which contains the ampicillin resistance gene.

Plasmid RSV-Red2 contains an RSV promoter, a lac promoter driving the AsRed2 product (Lac-AsRed2), flanked by an I-SceI and PI-PspI site, followed by an SV40 PolyA. This pRSV-Red2 plasmid was constructed by cloning the RSV promoter into a plasmid constructed from pUC19 and pkRFP (Clontech). The pSL1180-Pan9-Avr(II) plasmid was digested by NruI (FIG. 1B) to release the Pan9 E3 fragment, pRSV-Red2 was digested with PvuII and HpaI (FIG. 1C) to release the I-SceI-RSV-lac-AsRed2-PI-SceI cassette and religated to form a plasmid SL1180-Pan9-Avr(II)pkRFP, which contains the ampicillin resistance gene, and the I-SceI-RSV-lac-AsRed2-PI-SceI cassette in the site of the E3 region.

Also resulting from the AvrII digestion of pPan9-pkGFP described above is a plasmid containing the Pan9 viral genome having a deletion in the E3 region (FIG. 1D); following religation, the resulting plasmid is termed pPan9-pkGFP-Avr(II). This plasmid and the pSL1180-Pan9-Avr(II)pkRFP plasmid are digested with AvrII (FIG. 1E) and religated to form a single plasmid containing the GFP reporter gene in the E1 locus and the RFP reporter gene in the E3 locus. The resulting plasmid is termed pPan9-(E1)-pkGFP-(E3)pkRFP. By selecting for colonies expressing yellow, one selects for vectors expressing both the green and red fluorescent proteins.

This design allows easy shuttle in the antigen expression cassettes and convenient color-based colony selection of recombinants, which will be suitable for high throughput vector creation.

B. Construction of Polyvalent Transfer Vector

In order to construct a polyvalent transfer vector of the invention, a first selected heterologous expression cassette is cloned into an appropriate site in a shuttle vector.

With reference to FIG. 2, this is illustrated by cloning “antigene 1” into a plasmid having multiple cloning site, selecting the site flanked by restriction enzyme sites corresponding to those in the desired region of the DNA molecule produced according to part A above, i.e., flanked by I-CeuI and PI-SceI sites.

A second heterologous expression cassette (antigene 2) is cloned into an appropriate vector (e.g., pUC19-RSV) between I-SceI sites.

Following digestion with the appropriate enzymes, vectors containing the desired heterologous expression cassettes can be selected by colorimetric means. More particularly, colonies expressing red upon excitation with the appropriate wavelength of light indicate the presence of vectors in which the movable cassette containing the GFP have been replaced with antigene 1, but the movable cassette containing the RFP has been retained. Colonies expressing green upon excitation with the appropriate wavelength of light indicate the presence of vectors in which the movable cassette containing the RFP has been replaced with antigene 1, but the movable cassette containing the GFP has been retained. Colonies which appear white after excitation with light of the appropriate wavelengths for the GFP and RFP indicate vectors in which both movable cassettes have been replaced with heterologous expression cassettes. Thus, by selecting for the white colonies, one can rapidly and accurately select for polyvalent transfer vectors of the invention.

C. Study of Impact of Location (E1 or E3) and Orientation of Transgene Cassettes on Antigen Expression

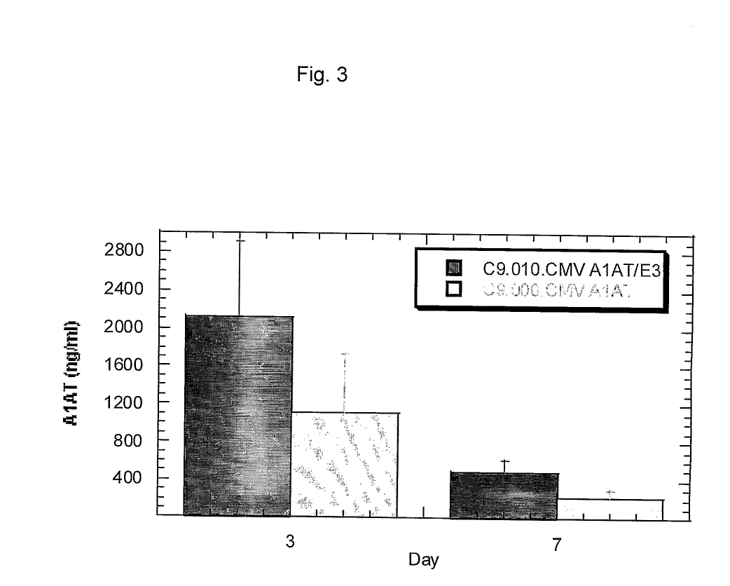

Identical CMV-hA1AT expression cassettes were cloned into E1 locus of E1-deleted Pan9 (also termed C9) vector and E3 locus of E1/E3-deleted C9 vector separately. The recombinant C9 viruses expressing A1AT from different loci were produced and injected into C57BL/6 mice intravenously at a dose of 1×10¹¹ pts/mouse. The animals were bled at days 3 and 7 post gene transfer and serum hA1AT levels were measured for comparison. The data revealed that hA1AT expressed from E3 locus were actually at least 2 folds higher than that from E1 locus. See, FIG. 3.

The cloning process of the invention has a convenient dual color selection system for the recombinants of mono- or polyvalent vector. Further, this data demonstrates that there is no negative locus dependent effect on the transgene expression in simian adenovirus vectors.

All publications cited in this specification, and the sequence listing, are incorporated herein by reference. While the invention has been described with reference to particular embodiments, it will be appreciated that modifications can be made without departing from the spirit of the invention. Such modifications are intended to fall within the scope of the appended claims.

1. A polyvalent plasmid backbone comprising:
(a) a first movable cassette located in a first locus of a viral genome, said first movable cassette comprising nucleic acid sequences comprising a first detectable reporter gene operably linked to sequences that will direct expression thereof, said movable cassette being flanked by a first set of rare restriction enzyme sites composed of two rare restriction enzyme sites; and
(b) a second movable cassette located in a second locus of the viral genome, said second movable cassette comprising nucleic acid sequences comprising a second detectable reporter gene operably linked to sequences that will direct expression thereof, said movable cassette being flanked by a second set of rare restriction enzyme sites composed of two rare restriction enzyme sites;
wherein the product of said first detectable reporter gene and the second detectable reporter gene are distinguishable, and
wherein the first and second set of rare restriction enzyme sites differ.

2. The polyvalent plasmid backbone according to claim 1, further comprising three or more loci.

3. The plasmid backbone according to claim 1, wherein the first and second locus are located in different gene regions within the viral genome.

4. The plasmid backbone according to claim 1, wherein the first and second locus are located in different open reading frames within a single gene region of the viral genome.

5. The plasmid backbone according to claim 1, wherein the first and second locus are located within a single open reading frame of a gene region of the viral genome and are non-contiguous with one another.

7. The plasmid backbone according to claim 1, wherein the detectable reporter genes are differentiated from one another by product color.

8. The plasmid backbone according to claim 1, wherein the detectable reporter genes are independently selected from the group consisting of green fluorescent protein, red fluorescent protein, and beta-galactosidase.

9. The plasmid backbone according to claim 1, wherein the viral genome is an adenoviral genome.

10. The plasmid backbone according to claim 1, wherein the viral genome is selected from the group consisting of a lentiviral genome, retroviral genome, and a poxvirus genome.

11. The plasmid backbone according to claim 1, wherein the two rare restriction enzyme sites in the first set are independently selected from the group consisting of I-Ceu I, PI-Sce I, TevII, BmoI, and DmoI, and differ from one another.

12. (canceled)

13. (canceled)

14. The method according to claim 13, wherein the first detectable reporter gene is green fluorescent protein and the second detectable reporter gene is red fluorescent protein.

15. The method according to claim 13, wherein the first set of enzymes is I-Ceu I and PI-Sce I.

16. The method according to claim 13, wherein the second set of enzymes differs from the first set of enzymes.

17. The method according to claim 13, wherein the polyvalent transfer vector comprises sequences from a viral genome selected from the group consisting of an adenovirus, a lentivirus, a retrovirus, a poxvirus.

18. The method according to claim 16, wherein the polyvalent transfer vector comprises adenoviral sequences in which the first expression cassette is located in an adenovirus E1 region.

19. The method according to claim 16, wherein the polyvalent transfer vector comprises adenoviral sequences in which the second expression cassette is located in an adenovirus E4 region.

20. The method according to claim 16, wherein the polyvalent transfer vector comprises adenoviral sequences in which the second expression cassette is located in the adenovirus E3 region.

21. (canceled)

22. (canceled)

23. A method of generating a polyvalent virus comprising the step of culturing the polyvalent transfer vector prepared according to claim 36 in the presence of sufficient viral sequences to permit packaging of the polyvalent viral genome into an infectious viral envelope.

24. A polyvalent virus produced according to the method of claim 23.

25. The polyvalent virus according to claim 24, wherein said virus is packaged in an infectious envelope comprising a lentivirus envelope protein, a retrovirus envelope protein, and a poxvirus envelope protein.

26. The polyvalent virus according to claim 25, wherein the poxvirus is canarypox.

27. A method of generating a polyvalent virus comprising the step of culturing the polyvalent plasmid backbone according to claim 13 in the presence of sufficient viral sequences to permit packaging of the polyvalent viral backbone into an infectious viral capsid.

28. A polyvalent viral vector produced according to the method of claim 27.

29. A polyvalent viral vector according to claim 28 comprising an adenoviral capsid protein.

30. A composition containing:
(a) a polyvalent viral vector comprising:
(i) a first heterologous expression cassette comprising a nucleic acid sequence encoding a first target product under the control of regulatory sequence that control expression of the product; and
(ii) a second heterologous expression cassette comprising a nucleic acid sequence encoding a second target product under the control of regulatory sequence that control expression of the product;
wherein said first and second expression cassettes are located in distinct loci of a viral vector genome and the target products are independently expressed; and
(b) a physiologically compatible carrier.

31. The composition according to claim 30, wherein said first and second products are independently selected from the group consisting of an antigen and a cytokine.

32. The composition according to claim 30, wherein the first or second product is an immune modulator.

33. The composition according to claim 32, wherein the second product is an adjuvant for the first product.

34. The composition according to claim 32, wherein the first or second product is a therapeutic gene product.

35. A cell culture comprising cells containing the polyvalent plasmid backbone according to claim 1.

36. A method for generating a polyvalent transfer vector, said method comprising the steps of:
(a) mixing, in the presence of the first set of enzymes, a polyvalent plasmid backbone according to claim 1 and a first nucleic acid molecule comprising a first heterologous expression cassette comprising a nucleic acid sequence encoding a target product under the control of regulatory sequence that control expression of the product, wherein said heterologous expression cassette is flanked by the first set of restriction enzyme sites;
(b) selecting for the absence of the first detectable reporter to provide plasmid backbones containing the first heterologous expression cassette;
(c) mixing, in the presence of the second set of enzymes, the polyvalent plasmid backbone and a second nucleic acid molecule comprising a second heterologous expression cassette comprising a nucleic acid sequence encoding a target product under the control of regulatory sequence that control expression of the product, wherein said heterologous expression cassette is flanked by the second set of rare restriction enzyme sites; and
(d) selecting for the absence of the second detectable reporter to provide plasmid backbones containing the second heterologous expression cassette,
thereby providing a polyvalent transfer vector containing a polyvalent viral genome comprising a first expression cassette in a first locus and a second expression cassette in a second locus.

37. A polyvalent transfer vector produced according to the method of claim 13.
